(19) United States
(12) Patent Application Publication
(10) Pub. No.: US 2022/0162549 A1
(43) Pub. Date: May 26, 2022

(54) METHOD OF HARVESTING MESENCHYMAL STEM CELLS FROM CORD-PLACENTA JUNCTION

(71) Applicant: Naimisha Beeravolu, Monmouth Junction, NJ (US)

(72) Inventor: Naimisha Beeravolu, Monmouth Junction, NJ (US)

(21) Appl. No.: 16/953,709

(22) Filed: Nov. 20, 2020

(51) Int. Cl. C12N 5/073

(52) U.S. Cl. CPC C12N 5/0605; C12N 2506/1346

(57) ABSTRACT

The present invention is a method of harvesting mesenchymal stem/stromal cells (MSCs) from human umbilical cord/placenta tissue. A key aspect of the invention is that MSCs are harvested from a specific region of the human umbilical cord/placenta, namely, the cord placenta junction (CPJ). In accordance with the method, the tissue is divided into distinct regions, one of which is the cord placenta junction (CPJ). MSCs from the CPJ are isolated and cultured. Harvesting MSCs from the CPJ yields a higher number of cells, with greater proliferation and self-renewal capacities in vitro.

### FIELD OF THE INVENTION

The present invention relates to human umbilical cord mesenchymal stem/stromal cells. More particularly, the invention discloses a method of efficiently harvesting human umbilical cord placenta junction derived mesenchymal stem/stromal cells for therapeutic use as well as for in vitro 3D model system for screening applications.

### BACKGROUND OF THE INVENTION

Human umbilical cord mesenchymal stem/stromal cells (HUCMSCs) have emerged as a source of mesenchymal stem/stromal cells (MSCs), and they have many advantages over bone marrow-derived MSCs (BMSCs). The harvesting procedure is noninvasive, and umbilical cords are discarded tissues. Additionally, HUCMSCs strongly express MSC surface markers similar to BMSCs. HUCMSCs are negative for hematopoietic markers such as cluster of differentiation (CD) 34 and CD45. Also, HUCMSCs are capable of differentiating into mesenchymal lineages, and the number of fibroblast colony-forming units is significantly higher in HUCMSCs than in BMSCs. HUCMSCs are capable of chondrogenic differentiation in vitro and in vivo even better than BMSCs. HUCMSCs also have anti-apoptotic potential. These findings indicate that HUCMSCs may be a potential stem cell source for various therapeutic applications.

The human umbilical cord (UC) and placenta are non-invasive, primitive and abundant sources of mesenchymal stem/stromal cells (MSCs) that have increasingly gained attention because they do not pose any ethical or moral concerns. Current methods to isolate MSCs from the UC and placenta yield low amounts of cells with variable proliferation potentials. Since the UC is an anatomically-complex organ, differences in MSC properties may be due to the differences in the anatomical regions of their isolation.

There are many possible therapeutic applications for UC derived MSCs including:

### Treatment of Degenerative Diseases

Degenerative Diseases such as Intervertebral disc (IVD) degeneration and Osteoarthritis are characterized by the loss of nucleus pulposus (NP) and wear and tear of the cartilage tissue, respectively, which is a common cause for pain. Although, currently, there is no cure for the degenerative diseases, stem cell therapy could be considered for its potential treatment. Pre-clinical studies have proven the feasibility and efficacy of human umbilical cord placenta junction derived mesenchymal stem cells (CPJ-MSCs) and chondroprogenitor cells (CPCs) derived from those cells to regenerate damaged IVD in a rabbit model. Moreover, the chondroprogenitor cells demonstrated to be more efficacious.

### Testing of Radiocontrast Dyes

Radiocontrast dyes are used for a wide range of diagnostic procedures for enhancing the image of anatomical structures, pain targets, and vascular uptake. However, their safety/toxicity on the primary cells and stem cells in the human body is unclear. Therefore, the human umbilical cord placenta junction derived mesenchymal stem cells can be used as a model system to test cytotoxicity of various diagnostic dyes prior to their usage in the field of diagnostic world. This will aid medical practitioners to pick the safe product for their patient's better health.

### Human Umbilical Cord Blood and Tissue Banking

Human Umbilical Cord Blood and Tissue banking has been extensively practiced lately upon awareness of the potential of stem cells and their applications to treat various diseases. Process and storage of cord tissue has been still a challenge as it occupies more space and the best storage method has not yet been optimized/studied to evaluate future stability of the stored sample. Since my work has shown that cord placenta junction is the most potential site in the whole human umbilical cord anatomically, it could be the ideal tissue that could be stored/banked for future use.

MSCs from umbilical cord were commonly harvested from a whole tissue or from separated regions such as cord tissue (CT), Wharton's jelly(WJ) and fetal placenta (FP). In accordance with the method of the invention, MSCs are harvested exclusively from the CPJ and were compared with CT, WJ, and FP.

Harvesting MSCs from the CPJ yields a higher number of cells, with greater proliferation and self-renewal capacities in vitro. Accordingly, the CPJ is a more promising source of MSCs for cell therapy, regenerative medicine, and tissue engineering.

The present invention is a method of harvesting mesenchymal stem/stromal cells (MSCs) from human umbilical cord/placenta tissue. A key aspect of the invention is that MSCs are only harvested from a specific region of the human umbilical cord/placenta, namely, the cord placenta junction (CPJ). In accordance with the method, the tissue is divided into distinct regions, one of which is the cord placenta junction (CPJ). MSCs from the CPJ are isolated and cultured. Harvesting MSCs from the CPJ yields a higher number of cells, with greater proliferation and self-renewal capacities in vitro.

### SUMMARY OF THE INVENTION

It is a major object of the invention to provide a method of harvesting human umbilical cord mesenchymal stem/stromal cells (MSCs).

It is another object of the invention to provide a method of harvesting MSCs that provides an increased yield.

It is another object of the invention to provide a method of harvesting MSCs that produces cells with greater proliferation.

It is another object of the invention to provide a method of harvesting MSCs that produces cells with greater self-renewal and targeted differentiation properties.

### BRIEF DESCRIPTION OF THE DRAWINGS

FIG. 1 shows a diagrammatic representation of the method of the invention.

### DETAILED DESCRIPTION

Human umbilical cord (UC) blood and tissue are non-invasive sources of potential stem/progenitor cells with similar cell surface properties as bone marrow stem/stromal cells (BMSCs). However, the UC is an anatomically complex organ and the potential of cells in various sites of the UC has not been fully explored. Commonly dissected regions from the UC to isolate stem cells are cord tissue (CT), Wharton's jelly (WJ) and Fetal Placenta (FP). The idea of separating the cord placenta junction (CPJ) as a distinct anatomical region of the human umbilical cord and isolating the perinatal/mesenchymal stem cells is a key aspect of the invention as will be described in more detail below. In prior art attempts at harvesting MSCs, cells are generally harvested from all regions of the UC. The present invention lies in the discovery that the CPJ is the most potent anatomical region of the UC and yields a higher number of cells, with greater proliferation and self-renewal capacities in comparison to the other sites of the human umbilical cord. Therefore, pre-differentiation of the CPJ derived mesenchymal stem cells into the progenitors of target tissue lineage would pose far greater capacity to regenerate and treat degenerative diseases such as Osteoarthritis and Intervertebral Disc Disease.

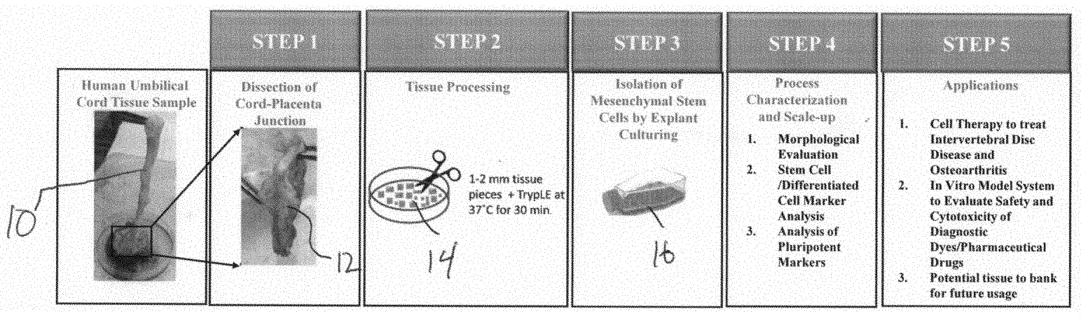

In accordance with the method of the invention the cord/placenta samples 10 are dissected and the CPJ 12 is isolated as can be seen in FIG. 1. The dissected sample 12 is then cut into sections 14. The sections 14 can be square with sides of between 1 to 2 mm.

The explant culture technique is then used to isolate cells from the CPJ in a suitable growth medium 16. Outgrowth of the cells from the CPJ occur within 3-4 days, and by experimentation it has been discovered that this is faster than cells from the other UC sites. The isolated cells are adherent to plastic and display fibroblastoid morphology and surface markers, such as CD29, CD44, CD73, CD90, and CD105, similarly to bone marrow (BM)-derived MSCs. MSCs from the CPJ differentiate into adipogenic, chondrogenic, and osteogenic lineages, indicating that they were multipotent (as do MSCs from the other sources). However, when compared to MSCs from the other UC sites, as well as (BM)-derived MSCs, experimental data indicates CPJ-MSCs differentiate more efficiently in comparison to other MSC sources. Accordingly, it has been determined that the CPJ is the most potent anatomical region and yields a higher number of cells, with greater proliferation and self-renewal capacities in vitro. A comparative analysis of the MSCs from the four sources indicated that the CPJ is a more promising source of MSCs for cell therapy, regenerative medicine, and tissue engineering.

I claim:

1. A method of harvesting mesenchymal stem/stromal cells from human umbilical cord/placenta tissue comprising the steps:
isolating and dissecting the cord placenta junction from the cord/placenta tissue;
cutting the cord placenta junction tissue into sections; and,
isolating the mesenchymal stem/stromal cells by explant culturing in a growth medium.

2. The method of claim 1 wherein said sections are substantially square.

3. The method of claim 2 wherein sides of said sections are between 1 and 2 mm.

4. The method of claim 1 wherein duration of said explant culturing step is about 3 or 4 days.

5. The method of claim 1 including the step of removing said mesenchymal stem/stromal cells from said growth medium.
